(19) United States
(12) Patent Application Publication
(10) Pub. No.: US 2004/0043486 A1
(43) Pub. Date: Mar. 4, 2004

(54) Methods and compositions for use in homologous recombination

(75) Inventor: Patrick Fogarty, Santa Cruz, CA (US)

Correspondence Address: BOZICEVIC, FIELD & FRANCIS LLP, 200 MIDDLEFIELD RD, SUITE 200, MENLO PARK, CA, 94025, US

(73) Assignee: Tosk, Inc.

(21) Appl. No.: 10/446,635

(22) Filed: May 27, 2003

Related U.S. Application Data
(63) Continuation-in-part of application No. 10/427,551, filed on Apr. 30, 2003.
(60) Provisional application No. 60/377,026, filed on Apr. 30, 2002.
(60) Provisional application No. 60/396,992, filed on Jul. 18, 2002.

(51) Int. Cl. A61K 48/00; C12N 15/85; C12N 15/74

(52) U.S. Cl. 435/455; 424/93.21; 435/473

(57) ABSTRACT

Methods for homologously recombining an exogenous nucleic acid into a target cell genome of a target cell are provided. In the subject methods, a targeting vector that includes a linearizing endonuclease site, e.g., a recombinase recognition site, and a homologous recombination integrating element, is contacted with the target cell(s) such that the target cell(s) take up the targeting vector. The targeting vector is originally a circular targeting vector that is linearized by a linearizing endonuclease, e.g., a recombinase, either prior to administration (e.g., in vitro treatment with an endonuclease) or upon entry into the multicellular organism by endonucleases in the extracellular or intracellular fluids that can be introduced to or already present in the muticellular organism. The integrating element homologously recombines into the target cell genome from the linearized targeting vector. Also provided are targeting vectors, systems and kits for use in practicing the subject methods.

### CROSS-REFERENCE TO RELATED APPLICATIONS

This application is a continuation-in-part of application Ser. No. 10/427,551 filed on Apr. 30, 2003, which application pursuant to 35 U.S.C. §119 (e), claims priority to the filing date of the U.S. Provisional Patent Application Serial No. 60/377,026 filed Apr. 30, 2002 and to the filing date of U.S. Provisional Patent Application Serial No. 60/396,992 filed Jul. 18, 2002; the disclosures of which are herein incorporated by reference.

### INTRODUCTION

1. Field of the Invention

The field of this invention is nucleic acid integration, and more specifically homologous recombination.

2. Background of the Invention

Reagents and methods that facilitate directed genome modification in multicellular organisms constitute a powerful toolbox for experimental genetics, human therapeutics and agriculture. One strategy for genomic modification that finds use is the direct alteration of cellular genomes by gene targeting, in which an exogenous DNA undergoes homologous recombination with its corresponding chromosomal site in a target cell. Gene targeting technologies have the advantage that the introduced gene resides at its normal chromosomal locus and, as such, the major problems e.g., gene silencing, insertional mutagenesis, ectopic gene expression and lack of stability, associated with alternative methods for gene introduction into cells, are avoided.

However in mammalian cells, only very low frequencies of homologous targeting events have been achieved, usually in the range of 10⁻⁶ per cell. In addition, homologous targeting occurs against a background of non-homologous events that are 100- to 1000-fold more common (Capecchi 1989 Science 244: 1288-1292). Many procedures have been devised to select or screen for the rate of successful targeting products, but the low absolute frequency of favorable events remains a serious limitation. As such, current gene targeting technologies, at least for mammalian cells, rely on the administration of linearized vectors to isolated cells or cells grown in tissue culture. Such cells may be used to create transgenic animals, and potentially may be used for transplant therapy. The need for specialized cell types greatly limits the utility of this gene targeting approach.

More effective transformation vectors are continually being produced, but these are primarily for cellular administration, primarily because of the dramatic difference in complexity when operating in a cell versus a whole animal.

As such, there is continued interest in the development of new transformation vectors. Of particular interest is the development of vectors that provide for stable site-specific integration of exogenous DNA into a cellular genome in an intact multicellular organism via homologous recombination.

Relevant Literature

References of interest include: U.S. Pat. Nos. 6,291,243; 5,719,055 and 4,670,388; as well as Rong and Golic (Science, 288: 2013-2018 2000); Rouet et al., (Proc. Natl. Acad. Sci. 91: 6064-6068, 1994); Segal et al., (Proc. Natl. Acad. Sci. 92: 806-810, 1995).

### SUMMARY OF THE INVENTION

Methods for homologously recombining an exogenous nucleic acid into a target cell genome of a target cell are provided. In the subject methods, a targeting vector that includes a linearizing endonuclease site, e.g., a recombinase recognition site, and a homologous recombination integrating element, is contacted with the target cell(s) such that the target cell(s) take up the targeting vector. The targeting vector is originally a circular targeting vector that is linearized by a linearizing endonuclease, e.g., a recombinase, either prior to administration (e.g., in vitro treatment with an endonuclease) or upon entry into the multicellular organism by endonucleases in the extracellular or intracellular fluids that can be introduced to or already present in the muticellular organism. The integrating element homologously recombines into the target cell genome from the linearized targeting vector. Also provided are targeting vectors, systems and kits for use in practicing the subject methods.

### BRIEF DESCRIPTION OF THE DRAWINGS

FIG. 1 is a schematic of the linearized ODC vector and the normal genomic ODC gene employed in the Experimental Section, below.

### DEFINITIONS

A “vector” is a double-stranded extrachromosomal nucleic acid that includes cloning and expression vehicles, as well as viral vectors. A “vector” is capable of transferring gene sequences to target cells. Typically, “vector construct,” “expression vector,” and “gene transfer vector,” mean any nucleic acid construct that can transfer gene sequences to target cells. Thus, the term includes cloning, and expression vehicles, as well as integrating vectors. A “circular” vector describes a vector that is made from a circular nucleic acid and may be in supercoiled or a relaxed form. A circular vector is not a linear or linearized vector.

“Exogenous nucleic acid” is a nucleic acid that, prior to practice of the subject methods, exists outside of a target cell. In certain embodiments, the exogenous nucleic acid is one that has a contiguous sequence that is not present in the genome of the target cell. In other embodiments, the exogenous nucleic acid has a contiguous sequence that is found in the genome of the target cell.

A “target site” is a predetermined location within a genome into which integration of an exogenous nucleic acid is desired.

A “homologous sequence” is a sequence that displays sequence identity to a “target site” for integration.

“Homologous recombination” is the integration of an integration element that includes an exogenous nucleic flanked by sequences that provide for homologous recombination into a target genome by a mechanism that is facilitated by there being a sufficiently high level of sequence identity, e.g., 75%, 80%, 85%, 90%, 95%, 98%, 99%, including 100% sequence identity, between the homologous flanking sequences of the integration element and the target site of the target genome. Homologous recombination results in the insertion into the target genomic site of the integration element that includes the homologous flanking sequences of the integration element.

“Gene targeting” describes the site specific integration of an exogenous nucleic acid into a specific target site of a target genome by homologous recombination.

By “nucleic acid fragment of interest” it is meant any nucleic acid fragment adapted for insertion into a genome. Suitable examples of nucleic acid fragments of interest include promoter elements, therapeutic genes, marker genes, control regions, trait-producing fragments, nucleic acid elements to accomplish gene disruption, and the like. A nucleic acid fragment of interest may additionally be an “expression cassette”, where an “expression cassette” comprises any nucleic acid construct capable of directing the expression of a gene/coding sequence of interest. A nucleic acid fragment of interest may also be a “disrupting” nucleic acid, where the disrupting nucleic acid, once integrated into a target site, will disrupt the expression of a gene in the vicinity of the target site e.g. the disrupting nucleic acid may alter the coding sequence of the gene, may interfere with the transcription, splicing or translation of the gene or may itself express a disruptive e.g. antisense nucleic acid.

Methods of transforming cells are well known in the art. By “transformed” it is meant an alteration in a cell resulting from the uptake of foreign nucleic acid, usually DNA. Use of the term “transformation” is not intended to limit introduction of the foreign nucleic acid to any particular method. Suitable methods include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. The nucleic acid molecule may be linear or circular.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

A “coding sequence” or a sequence which “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence.

“Operably linked” refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 85%-90%, more preferably at least about 90%-95%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

Two nucleic acid fragments are considered to “selectively hybridize” as described herein. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

A first polynucleotide is “derived from” a second polynucleotide if it has the same or substantially the same nucleotide sequence as a region of the second polynucleotide, its cDNA, complements thereof, or if it displays sequence identity as described above.

A first polypeptide is “derived from” a second polypeptide if it is (i) encoded by a first polynucleotide derived from a second polynucleotide, or (ii) displays sequence identity to the second polypeptides as described above.

“Substantially purified” general refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

An “endonuclease” describes any molecule capable of severing, internal (e.g., not at the 5′ or 3′ end of the DNA chain), the covalent linkage in a DNA chain of nucleotides, resulting in double stranded breaks at a particular sequence of a double stranded nucleic acid substrate, where the particular sequence is termed an “endonuclease site”. An “endonuclease” may be a DNAse, e.g., a restriction endonuclease, nickase, etc., or a recombinase, e.g. a transposase, resolvase, integrase, invertase etc.

A “restriction endonuclease” is a member of a family of enzymes that mediate site specific cleavage (i.e. at a specific DNA sequence) of double stranded nucleic acid molecules. Restriction endonucleases that are of interest are the so called “rare cutting” restriction endonucleases, which recognize and mediate cleavage at specific DNA sequences of at least 8 bases pairs in length. Rare cutting restriction endonucleases are discussed in Lamber et al (Mutat Res. 1999;433:159-68); Belford et al (Nucleic Acids Res 1997;25:3379-88) and Jasin, 1996 (Trends Genet. 12:244-228). Of particular interest is the I-Scel endonuclease (Dujon, Gene 1989 92-119), which recognizes an 18 bp non-palindromic sequence, and chimeric I-Scel nucleases (Bibikova et al., Mol. Cell. Bio. 2001 21: 289-287) and the HO endonuclease of Saccharomyces cerevisiae (encoded by NCBI accession number X90957).

A “recombinase” is a member of a family of enzymes that mediate site-specific recombination between specific DNA sequences recognized by the recombinase (Esposito, D., and Scocca, J. J., Nucleic Acids Research 25, 3605-3614 (1997); Nunes-Duby, S. E., et al., Nucleic Acids Research 26, 391-406 (1998); Stark, W. M., et al., Trends in Genetics 8, 432-439 (1992); Sadowski, 1993 FASEB J 7: 760-767). A recombinase may be a resolvase, integrase, invertase and transposase.

### DESCRIPTION OF THE SPECIFIC EMBODIMENTS

Methods for homologously recombining an exogenous nucleic acid into a target cell genome of a target cell are provided. In the subject methods, a targeting vector that includes a linearizing endonuclease site, e.g., a recombinase recognition site, and a homologous recombination integrating element, is contacted with the target cell(s) such that the target cell(s) take up the targeting vector. The targeting vector is originally a circular targeting vector that is linearized by a linearizing endonuclease, e.g., a recombinase, either prior to administration (e.g., in vitro treatment with an endonuclease) or upon entry into the multicellular organism by endonucleases in the extracellular or intracellular fluids that can be introduced to or already present in the muticellular organism. The integrating element homologously recombines into the target cell genome from the linearized targeting vector. Also provided are targeting vectors, systems and kits for use in practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In addition, many modifications may be made to adapt a particular situation, material, composition of matter, process, process step or steps, to the objective, spirit and scope of the present invention. All such modifications are intended to be within the scope of the claims made herein.

In this specification and the appended claims, the singular forms “a,” “an” and “the” include plural reference unless the context clearly dictates otherwise. Conversely, it is contemplated that the claims may be so-drafted to exclude any optional element. This statement is intended to serve as antecedent basis for use of such exclusive terminology as “solely,” “only” and the like in connection with the recitation of claim elements or by use of a “negative” limitation.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Also, it is contemplated that any optional feature of the inventive variations described herein may be set forth and claimed independently, or in combination with any one or more of the features described herein.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All existing subject matter mentioned herein (e.g., publications, patents, patent applications and hardware) is incorporated by reference herein in its entirety. The referenced items are provided solely for their disclosure prior to the filing date of the present application. Nothing herein is to be construed as an admission that the present invention is not entitled to antedate such material by virtue of prior invention.

As summarized above, the subject invention provides methods of homologously recombining an exogenous nucleic acid into a target cell genome of a target cell, as well as kits and systems for use in practicing the subject methods. In further describing the subject invention, the methods will be described first in greater detail, followed by a review of the subject systems and kits, as well as components thereof, for use in practicing the subject methods.

Methods of Using the Subject Gene Targeting Vectors

The subject vectors as described above find use in a variety of applications in which it is desired to introduce and stably integrate an exogenous nucleic acid into the genome of a target cell, e.g., a target cell present in a multicellular organism, such as a plant, animal, e.g., vertebrate, including mammal, etc. The subject vectors find particular use in the site specific integration via homologous recombination of exogenous nucleic acids into the genomes of target cells of animals, including insects, vertebrates, etc. In certain embodiments, the animals with which the subject vectors may be employed are vertebrates, where in many embodiments the animals are mammals. As such, of particular interest in many embodiments is the use of the subject vectors to target vertebrate animals, particularly avian and marine animals, e.g., chickens, zebrafish, and the like; mammalian animals, including murine, ungulate, porcine, ovine, equine, rat, dog, cat, monkey, humans, and the like.

In the methods of the subject invention, a targeting vector according to the subject invention is contacted with the target cell under conditions sufficient such that the targeting vector is taken up or internalized by the cell. The initially circular targeting vector is, prior to the homologous recombination event, linearized and the integrating element of the vector is inserted into the genome of the target cell by homologous recombination.

Targeting Vector

As indicated above, targeting vectors employed in the subject methods are circular vectors, and more specifically circular, double-stranded DNA vectors, i.e., plasmids. The size of the circular targeting vectors may vary, where the overall size may be at least about 100 base pairs, sometimes at least about 1000 base pairs and sometimes at least about 3 kb, where in certain embodiments the size may be as great as 300 kb or greater, but generally does not exceed about 30 kb and often does not exceed about 15 kb. The subject targeting vectors are vectors that include the following two elements: (a) a linearizing endonuclease site and (b) a homologous recombination integrating element. In certain embodiments, the targeting vectors may include one or more additional elements, such as linearizing endonuclease coding sequences, etc.

Linearizing Endonuclease Site

The subject targeting vectors include a linearizing endonuclease site. The linearizing endonuclease site is a site or domain of nucleotide residues that is recognized by an endonuclease, i.e., is cleaved by an endonuclease, such that the endonuclease enzymatically creates a double-stranded cleavage at the site, and thereby linearizes the circular targeting vector. The linearizing endonuclease site can be palindromic or non-palindromic, and is often at least about 4 nucleotides in length, sometimes at least about 16 nucleotides in length, sometimes at least about 24 nucleotides in length, where the length may be at least about 50 nucleotides or longer, but typically does not exceed about 600 nucleotides in length. The linearizing endonuclease site is generally one that is either not found in the target cell genome, exists in few copies so that when cleaved they are repaired within the cell such that no adverse consequences occur, or the cellular site(s) is protected, e.g., DNA structure (methylation, etc.), bound proteins, etc., so that practice of the subject methods does not result in significant cleavage of the target cell genome.

In certain embodiments, a feature of the subject vectors is that the linearizing endonuclease site is not selectively recognized by an endonuclease that is endogenous to the target cell. In other words, the target cell does not endogenously include a coding sequence for the linearizing endonuclease in its genome. As such, the linearizing endonuclease site is one that is recognized by a linearizing endonuclease that is endogenous to a species that is different from the species of the target cell. Any two given organisms are considered to be of different species if they are classified as such using standard taxonomical criteria, such as those employed to develop the taxonomy tables provided by the National Institutes of Health. Although the linearizing endonuclease is recognized by a linearizing endonuclease that is not endogenous to the target cell for it is designed, in certain embodiments the target cell will have been engineered to nonetheless express the linearizing endonuclease, as described in greater detail below. Another way of viewing this feature of the invention is that the linearizing endonuclease that recognizes its target site present in the targeting vector is one that is not found in the wild type organism of the target cell.

Depending on whether the targeting vector is to be employed in an “ends-out” or “ends-in” targeting scheme, the linearizing endonuclease site may be present inside of or outside of the integrating element, described in greater detail below. “Ends-out” and “ends-in” targeting schemes are known to those of skill in the art, and reviewed in Rong and Golic, Science (2000) 288: 20132018. As such, in certain embodiments, the linearizing endonuclease site is present inside of the integrating element. In yet other embodiments, the linearizing endonuclease site is present outside of the integrating element. In those embodiments where it is desired that the linearizing endonuclease site not be incorporated into the targeted genome, and ends-out approach is employed, such that the linearizing endonuclease site is positioned on the vector outside of the integrating element, i.e., either 5′ or 3′ to the integrating element on the vector.

The linearizing endonuclease site may be one that is recognized by restriction endonuclease, where particular endonucleases of interest are rare-cutter enzymes as described above, e.g., I-Sce-1, I-Ssp-1, etc., where in certain embodiments the site is recognized by a recombinase, where a recombinase may be a resolvase, integrase, invertase or transposase. Examples of recombinase and transposase systems of interest are, for example, the Cre-lox system from bacteriophage P1, the FLP-FRT system of Saccharomyces cerevisiae, the R-RS system of Zygosaccharomyces rouxii, the Gin-gix system of bacteriophage Mu, the P element transposase/P foot system of Drosophila melanogastar, the φC31 att site/integrase system. Where the endonuclease is a recombinase and the endonuclease site is a terminal repeat marking the boundaries of a transposon, the endonuclease and endonuclease site may be those from any one of several known transposable elements, such as, for example, the hobo, copia, gypsy elements of Drosophila, the Ac/Dc, En/Spm, Tam systems of plants, any of the Tc elements of C. elegans, the TY element of S. cerevisiae the mariner and mariner-like (e.g. Sleeping Beauty) elements of a variety of higher eukaryotes and any of the Tn and ISS transposable elements of E. coli. Where the endonuclease is a recombinase and the endonuclease site is a recombination site recognized by the endonuclease (e.g. an att site, a recognition sequence etc.), the endonuclease and endonuclease site can be derived from one of several bacteriophages, such as the λ, phiC31, γδ bacteriophage att systems, etc., transposable elements, viruses (e.g. adeno-associated virus, retroviruses etc.) or other systems. Given these examples, and definition of an endonuclease, one of skill in the art could readily identify other restriction endonucleases, recombinases, and transposases for use in this invention. Cleavage sites for these endonucleases, e.g. I-SceI and loxP sites, P foot, LTRs, att sites, etc., are well described in the art (Marshall et al TIG 1992 8:432-439; Hallet and Sherratt et al., FEMS Mic. Reviews 21 1999 157-178; Sadowski et al FASEB J, 1993 7:760-767.

In any event, the vector is linearized by any of the aforementioned methods or the like, either prior to or after its administration to the multicellular organism. The linearized vector is a key signaling event in the cell to initiate homologous recombination.

Integrating Element

In addition to the linearizing endonuclease site, the subject targeting vectors also contain a homologous recombination integrating element, which is made up of an exogenous nucleotide or nucleic acid (i.e., a nucleic acid that is desired to be integrated into the target cell genome) flanked by sequences that provide for homologous recombination with the target cell genome for which the vector is designed to be employed. As discussed above, homologous flanking sequences display sequence identity to a target site in the target genome, and facilitate homologous recombination between the integrating element of the vector and the target site to result in the insertion of the integrating element into the target cell genome. The length of the flanking homologous sequences may vary, where the length is typically at least about 500 base pairs, usually at least about 500 base pairs and more usually at least about 1 kb, where in certain embodiments the length is at least about 30 kb or longer, but sometimes does not exceed about 10 kb and sometimes does not exceed about 3 kb.

Flanked by the sequences that provide for homologous recombination is an exogenous nucleic acid that is to be inserted into the genome, where the exogenous nucleic acid is at least about 1 nt long, where in many embodiments the exogenous nucleic acid is at least two or more nt long, where in certain embodiments the exogenous nucleic acid is at least about 100 nt long, sometimes at least about 1,000 nt long, where the exogenous nucleic acid may be as long as 30 kb or longer, but sometimes does not exceed about 3,000 nt long. In certain embodiments, the exogenous nucleic acid includes an expression cassette, an expression modulatory nucleic acid, a therapeutic gene, an expression disrupting nucleic acid, and the like, where the nucleic acid fragment of interest may be a trait producing nucleic acid.

Optional Components

As indicated above, the subject vectors may also include a number of optional components, depending on the particular application or protocol for which the targeting vector has been designed. As described in greater detail below, in practicing the subject methods, in addition to the targeting vector, the endonuclease component (e.g., protein or nucleic acid encoding the same) of the subject systems is also introduced into the target cell, such that the endonuclease is present to mediate integration of the targeting vector into the target genome via homologous recombination. The linearizing endonuclease may be introduced into the target cell as a polypeptide or a nucleic acid that encodes a product having the desired endonuclease activity. In these latter embodiments, the nucleic acid may be introduced into the target cell in a vector separate from the targeting vector, or the nucleic acid encoding the desired endonuclease activity may be present on the targeting vector itself. As such, in these latter embodiments, the targeting vector further includes a domain that encodes for an endonuclease that recgonizes the linearizing endonuclease site on the vector. Endonuclease-encoding nucleic acids may be chemically synthesized or isolated from a host carrying the nucleic acid by conventional recombinant DNA practices (e.g. polymerase chain reaction, library screening etc.) and cloned into the targeting vector for expression. It is understood that such nucleotides may be modified (e.g. to remove restriction sites, to change codon usage, to change regulatory regions, to add/remove introns etc.) before use.

As indicated above, a linearizing endonuclease may be a restriction endonuclease or a recombinase, where a recombinase may be a resolvase, integrase, invertase or transposase. Examples of recombinase and transposase systems of interest are, for example, the Cre-lox system from bacteriophage P1, the FLP-FRT system of Saccharomyces cerevisiae, the R-RS system of Zygosaccharomyces rouxii, the Gin-gix system of bacteriophage Mu, the P element transposase/P foot system of Drosophila melanogastar, the φC31 att site/integrase system. Where the endonuclease is a recombinase and the endonuclease site is a terminal repeat marking the boundaries of a transposon, the endonuclease and endonuclease site may be those from any one of several known transposable elements, such as, for example, the hobo, copia, gypsy elements of Drosophila, the Ac/Dc, En/Spm, Tam systems of plants, any of the Tc elements of C. elegans, the TY element of S. cerevisiae the mariner and mariner-like (e.g. Sleeping Beauty) elements of a variety of higher eukaryotes and any of the Tn and ISS transposable elements of E. coli. Where the endonuclease is a recombinase and the endonuclease site is a recombination site recognized by the endonuclease (e.g. an att site, a recognition sequence etc.), the endonuclease and endonuclease site can be derived from one of several bacteriophages, such as the λ, phiC31, γδ bacteriophage att systems, etc., transposable elements, viruses (e.g. adeno-associated virus, retroviruses etc.) or other systems. As such, in certain embodiments the targeting vector includes a nucleic acid encoding a linearizing endonuclease, as described above. Furthermore, certain embodiments may use a system where the endonuclease is already present in the genome of the multicellular organism, either as a native gene or a genetically engineered organism.

Additional Optional Components

In certain embodiments, a selectable marker is further included in the gene targeting vector as a nucleic acid fragment of interest. The selectable marker may allow selection of cells containing an integrated gene targeting vector over cells that do not have an integrated vector.

Vector Construction

The vectors of the subject invention may be produced by standard methods of restriction enzyme cleavage, ligation and molecular cloning. One protocol for constructing the subject vectors includes the following steps. First, purified nucleic acid fragments containing desired component nucleotide sequences as well as extraneous sequences are cleaved with restriction endonucleases from initial sources. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g. sequences corresponding to the various elements of the subject vectors, as described above is produced. Where desired, the circular molecules so constructed are then amplified in a prokaryotic host, e.g. E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc.

Targeting Vector Contact with the Target Cell

As summarized above, in practicing the subject methods, a targeting vector of the subject invention is contacted with a target cell whose genome is to be modified, such that the targeting vector is internalized by the cell. Upon internalization of the targeting vector, (where at some point the targeting vector is linearized by an appropriate linearizing endonuclease), the integrating element is permitted to homologously recombine into the target cell genome.

Contact of the targeting vector with the target cell(s) may be accomplished using any convenient protocol. In those embodiments where the target cells are present as part of a multicellular organism, e.g., an animal, the targeting vector is typically administered to (e.g., injected into, fed to, etc.) the multicellular organism, e.g., a whole animal, where administration may be systemic or localized, e.g., directly to specific tissue(s) and/or organ(s) of the multicellular organism.

The targeting vector may be introduced into the animal cells using any convenient protocol, where the protocol may provide for in vivo, in vitro, or ex vivo introduction of the vector.

For example, where the target cell is an isolated cell, the targeting vector may be introduced directly into the cell under cell culture conditions permissive of viability of the target cell, e.g., by using standard transformation techniques. Such techniques include, but are not necessarily limited to: viral infection, transfection, conjugation, protoplast fusion, electroporation, particle gun technology, calcium phosphate precipitation, direct microinjection, viral vector delivery, and the like.

The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

Alternatively, where the target cell or cells are part of a multicellular organism, the targeting vector may be administered to the organism or host in a manner such that the targeting construct is able to enter the target cell(s), e.g., via an in vivo or ex vivo protocol. By “in vivo,” it is meant in the target construct is administered to a living body of an animal. By “ex vivo” it is meant that cells or organs are modified outside of the body. Such cells or organs are typically returned to a living body. Methods for the administration of nucleic acid constructs are well known in the art. Nucleic acid constructs can be delivered with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995,), using viral vectors (Monahan, et al, Gene Therapy 4:40-49, 1997; Onodera, et al, Blood 91:30-36, 1998,), by uptake of “naked DNA”, and the like. Techniques well known in the art for the transfection of cells (see discussion above) can be used for the ex vivo administration of nucleic acid constructs. The exact formulation, route of administration and dosage can be chosen empirically. (See e.g. Fingl et al., 1975, in “The Pharmacological Basis of Therapeutics”, Ch. 1 p1).

As such, in vivo protocols that find use in delivery of the subject vectors include delivery via lipid based, e.g. liposome vehicles, where the lipid based vehicle may be targeted to a specific cell type for cell or tissue specific delivery of the vector. Patents disclosing such methods include: U.S. Pat. Nos. 5,877,302; 5,840,710; 5,830,430; and 5,827,703, the disclosures of which are herein incorporated by reference. Other in vivo delivery systems may also be employed, including: the use of poly-lysine based peptides as carriers, which may or may not be modified with targeting moieties, microinjection, electroporation, and the like. (Brooks, A. I., et al. 1998, J. neurosci. Methods V. 80 p: 137-47; Muramatsu, T., Nakamura, A., and H. M. Park 1998, Int. J. Mol. Med. V. 1 p: 55-62). In certain embodiments of interest, viral based nucleic acid introduction vectors are not employed. Because of the multitude of different types of vectors and delivery vehicles that may be employed, administration may be by a number of different routes, where representative routes of administration include: oral, topical, intraarterial, intravenous, intraperitoneal, intramuscular, intranasal, intraconjunctival etc. The particular mode of administration depends, at least in part, on the nature of the delivery vehicle employed for the vectors. In many embodiments, the vector or vectors are administered intravascularly, e.g. intraarterially or intravenously, employing an aqueous based delivery vehicle, e.g. a saline solution.

The amount of vector nucleic acid that is introduced into the animal is sufficient to provide for the desired integration of the exogenous nucleic acid into the genome. As such, the amount of targeting vector nucleic acid introduced should provide for a sufficient copy number of the exogenous nucleic acid. The amount of vector nucleic acid that is introduced into the animal varies depending on the efficiency of the particular introduction or transfection protocol that is employed.

As indicated above, the targeting vectors are introduced into the target cells under conditions sufficient that provide for cleavage of the vector at the endonuclease site to linearize the initial circular targeting vector, i.e., vector linearization conditions, where such conditions are provided in a number of ways, where such conditions include situations where the initially circular targeting vector is linearized prior to administration to the multicellular organism and situations where the initially circular targeting vector is administered to the multicellular organism in its original circular format and then linearized in vivo, e.g., by employing a host that has been engineered to express the linearizing endonuclease in the target cell or co-administering the linearizing endonuclease or a coding sequence therefore to the cell or host containing the same, where co-administration may occur either before, after or at the same time as administration of the vector.

As such, in these latter embodiments where linearization of the initially circular targeting vector occurs in vivo, the endonuclease component (e.g., protein or nucleic acid encoding the same) of the subject systems is present in the target cell to mediate linearization of the targeting vector and integration of the integrating element thereof into the target genome via homologous recombination. As indicated above, the host may be pre-engineered to express the linearizing endonuclease, or the linearizing endonuclease may be administered to the target cell. Where the linearizing endonuclease activity is provided as polypeptide, any convenient polypeptide/protein introduction protocol may be employed. Methods of introducing functional proteins into cells are well known in the art. Alternatively, a nucleic encoding the endonuclease can be included in an expression vector used to transform the cell. Endonuclease-encoding nucleic acids may be chemically synthesized or isolated from a host carrying the nucleic acid by conventional recombinant DNA practices (e.g. polymerase chain reaction, library screening etc.) and cloned into an appropriate vector for expression. It is understood that such nucleotides may be modified (e.g. to remove restriction sites, to change codon usage, to change regulatory regions, to add/remove introns etc.) before use.

The methods, as described above, result in integration of the homologous recombination integrating element into the target cell genome.

The above described integration methods can be used to stably integrate a wide variety of exogenous nucleic acids into a target cell. In many embodiments, the sequence of nucleotides present in the exogenous nucleic acid will be one that is not found in the genome of the target cell, i.e., it will be heterologous to the target cell. In other embodiments, the sequence of the exogenous nucleic acid may actually be one that is present in the target cell.

As indicated above, the subject systems can be used with a variety of target cells, where target cells in many embodiments are non-bacterial target cells, and often eukaryotic target cells, including plant and animal target cells, e.g., insect cells, vertebrate cells, particularly avian cells, e.g., chicken cells; mammalian cells, including murine, porcine, ungulate, ovine, equine, rat, dog, cat, monkey, and human cells; and the like.

Utility

The subject methods find use in a variety of applications in which the site specific integration of an exogenous nucleic acid into a target cell is desired. Applications in which the subject vectors and methods find use include: research applications, polypeptide synthesis applications and therapeutic applications. Each of these representative categories of applications is described separately below in greater detail.

Research Applications

Examples of research applications in which the subject methods find use include applications designed to characterize a particular gene. In such applications, the vector is employed either: 1) to insert a gene or coding sequence of interest into a target cell; 2) to delete a gene in part or in whole; or 3) to replace one or more specific nucleotides with different nucleotides in a gene and the resultant effect on the animal's phenotype is observed. In this manner, information about the gene's activity and the nature of the product encoded thereby can be deduced.

One can also employ the subject vectors to produce models in which, expression, including overexpression and/or misexpression of a gene of interest, is produced in a cell and the effects of this mutant expression pattern are observed. As such, one can employ the subject methods and compositions for the efficient and rapid production of specifically designed transgenic, knockout, or knockin animals, which can be employed as models for a variety of different conditions, as is known in the art.

Polypeptide Synthesis Applications

In addition to the above research applications, the subject methods and vectors also find use in the synthesis of polypeptides, e.g. proteins of interest. In such applications, a vector that includes a gene encoding the polypeptide of interest in combination with requisite and/or desired expression regulatory sequences, e.g. promoters, etc., (i.e. an expression module) is introduced into the target cell that is to serve as an expression host for expression of the polypeptide. Following introduction and subsequent stable integration into the target cell genome, the targeted host cell is then maintained under conditions sufficient for expression of the integrated gene. Once the transformed host expressing the protein is prepared, the protein is then purified to produce the desired protein comprising composition. Any convenient protein purification procedures may be employed, where suitable protein purification methodologies are described in Guide to Protein Purification, (Deuthser ed.) (Academic Press, 1990). For example, a lysate may be prepared from the expression host expressing the protein, and purified using HPLC, exclusion chromatography, gel electrophoresis, affinity chromatography, and the like.

In Vivo Protein Production

Recombinant cells of the present invention are useful, as populations of recombinant cell lines, as populations of recombinant primary or secondary cells, recombinant clonal cell strains or lines, recombinant heterogenous cell strains or lines, and as cell mixtures in which at least one representative cell of one of the four preceding categories of recombinant cells is present. Such cells may be used in a delivery system for treating an individual with an abnormal or undesirable condition which responds to delivery of a therapeutic product, which is either: 1) a therapeutic protein (e.g., a protein which is absent, underproduced relative to the individual's physiologic needs, defective or inefficiently or inappropriately utilized in the individual; a protein with novel functions, such as enzymatic or transport functions) or 2) a therapeutic nucleic acid (e.g., RNA which inhibits gene expression or has intrinsic enzymatic activity). In the method of the present invention of providing a therapeutic protein or nucleic acid, recombinant primary cells, clonal cell strains or heterogenous cell strains are administered to an individual in whom the abnormal or undesirable condition is to be treated or prevented, in sufficient quantity and by an appropriate route, to express or make available the protein or exogenous DNA at physiologically relevant levels. Representative therapeutic proteins of interest include, but are not limited to: factor VIII, factor IX, β-globin, low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides, and the like (where the above proteins are human proteins). Alternatively, a vector system as described above is administered directly to an organism, e.g., via an appropriate system or local route of administration, where the vector components enter one or more target cells and modulate transcription of a targeted genomic domain, e.g., to achieve a therapeutic purpose, e.g., expression of therapeutic protein that is not expressed prior to practice of the subject methods. A physiologically relevant level is one which either approximates the level at which the product is normally produced in the body or results in improvement of the abnormal or undesirable condition. For example, hGH, hEPO, human insulinotropin, hGM-CSF, hG-CSF, humanα-interferon, or human FSHβ can be delivered systemically in humans for therapeutic benefits. As such, the subject vector systems find use in therapeutic applications, in which the methods and vectors are employed to modulated transcription of a genomic domain, e.g., one that encodes a therapeutic protein, of a target cell, e.g., as may be performed in gene therapy applications. The subject vectors may be used to modulate transcription of a wide variety therapeutic proteins.

In Vitro Protein Production

Recombinant cells from human or non-human species according to this invention can also be used for in vitro protein production. The cells are maintained under conditions, as are known in the art, which result in expression of the protein. Proteins expressed using the methods described may be purified from cell lysates or cell supernatants in order to purify the desired protein. Proteins made according to this method include therapeutic proteins which can be delivered to a human or non-human animal by conventional pharmaceutical routes as is known in the art (e.g., oral, intravenous, intramuscular, intranasal or subcutaneous). Such proteins include hGH, hEPO, and human insulinotropin, hGM-CSF, hG-CSF, FSHβ or α-interferon. These cells can be immortalized, primary, or secondary cells. The use of cells from other species may be desirable in cases where the non-human cells are advantageous for protein production purposes where the non-human protein is therapeutically or commercially useful, for example, the use of cells derived from salmon for the production of salmon calcitonin, the use of cells derived from pigs for the production of porcine insulin, and the use of bovine cells for the production of bovine growth hormone.

As such, the subject methods find use in the synthesis of polypeptides, e.g. proteins of interest. In such applications, a vector that includes a transcriptional modulatory unit for a polypeptide of interest is introduced into the target cell that is to serve as an expression host for expression of the polypeptide. Following introduction and subsequent stable integration into the target cell genome, the targeted host cell is then maintained under conditions sufficient for expression of the gene that is now operably linked to the newly integrated transcriptional modulatory unit. Once the transformed host expressing the protein is prepared, the protein is then purified to produce the desired protein comprising composition. Any convenient protein purification procedures may be employed, where suitable protein purification methodologies are described in Guide to Protein Purification, (Deuthser ed.) (Academic Press, 1990). For example, a lysate may be prepared from the expression host expressing the protein, and purified using HPLC, exclusion chromatography, gel electrophoresis, affinity chromatography, and the like.

Therapeutic Applications

The subject vectors also find use in therapeutic applications, in which the methods and vectors are employed to stably integrate a therapeutic nucleic acid, e.g., gene or protein/factor coding sequence thereof, into the genome of a target cell, i.e., gene therapy applications. The subject vectors may be used to deliver a wide variety of therapeutic nucleic acids. Specific therapeutic genes for use in the treatment of genetic defect based disease conditions include genes encoding the following products: factor VIII, factor IX, β-globin, low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, omithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides, and the like.

The above list of proteins refers to mammalian proteins, and in many embodiments human proteins, where the nucleotide and amino acid sequences of the above proteins are generally known to those of skill in the art. Cancer therapeutic genes that may be delivered via the subject vectors include: genes that enhance the antitumor activity of lymphocytes, genes whose expression product enhances the immunogenicity of tumor cells, tumor suppressor genes, toxin genes, suicide genes, multiple-drug resistance genes, antisense sequences, and the like.

Direct administration of the subject gene targeting vector to animals has many therapeutic utilities, including the correction of the sequence of mutant genes linked to a disease or condition, activation of endogenous genes, disruption of toxic endogenous genes, introduction of exogenous genes, etc. One of skill in the art would recognize many utilities for this gene targeting system.

Systems

Also provided are systems for use in performing the subject methods. The subject systems include, at a minimum, a targeting vector as described above. Where the targeting vector does not provide for the linearizing endonuclease activity, the subject systems also typically include a source of linearizing endonuclease activity, e.g., a polypeptide having the desired activity or a nucleic acid encoding a product having the desired endonuclease activity.

Kits

Also provided are kits for use in practicing the subject methods. The subject kits at least include a targeting vector as described above and a corresponding endonuclease(s) or endonuclease-encoding nucleic acid, where this latter component may or may not be a separate component from the targeting vector The subject kits may further include other components that find use in the subject invention, e.g., buffers, delivery vehicles, etc.

The various components of the kit may be present in separate containers or certain compatible components may be pre-combined into a single container, as desired.

In addition to the above components, the subject kits will further include instructions for practicing the subject invention. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Vector Construction

The starting materials for the DNA vectors described below are readily available to those of skill in the art (e.g., Casper4 and P element transposase plasmids (Flybase), Ornithine Decarboxylase (ODC) rat genomic DNA (obtainable from ATCC and its website having an address formed by placing “www.” before and “.org” after “atcc”) Whey Associated Protein (WAP) mouse genomic DNA (also obtainable from the ATCC (www.atcc.org), Sleeping Beauty DNA (University of Minnesota) (see e.g., U.S. Pat. No. 6,489,458 for relevant sequence information, the disclosure of which is herein incorporated by reference), and plasmid with a single PI-SCEI cut site (New England Biolabs)). As Casper4 does not integrate into the genome of non-Drosophila species, it can be used as is for the tests for homologous integration or one P foot can be deleted from the vector using standard restriction digests and cloning procedures (leaving a single P foot cleavage site). As for the Sleeping Beauty element, which does integrate into a variety of species, one foot must be deleted. As each foot (transposase recognition sequence) is identical, either can be removed. Then the P element vector, Sleeping Beauty vector, or PI-SCEI single cut site containing plasmid has the desired WAP or ODC DNA cloned into it as described below using standard molecular biology procedures. The vectors of the subject invention may be produced by standard methods of restriction enzyme cleavage, ligation and molecular cloning.

One protocol for constructing the subject vectors includes the following steps. First, purified nucleic acid fragments containing desired component nucleotide sequences as well as extraneous sequences are cleaved with restriction endonucleases from initial sources. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g. sequences corresponding to the various elements of the subject vectors, as described above is produced. Where desired, the circular molecules so constructed are then amplified in a prokaryotic host, e.g. E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc).

### EXAMPLE 1

### Gene Targeting of the Omithine Decarboxylase Gene of Rat Using a Circular Gene Targeting Vector

To demonstrate this strategy is successful, a circular gene targeting vector was constructed . The circular vector contained a single Drosophila P element transposase cut site (P foot) and a fragment of altered rat genomic DNA. The rat ornithine decarboxylase gene (odc) was chosen because its entire sequence was known and it was commercially available. In order to assay the integration of the vector, three small deletions were made in the odc gene of the vector. The small deletions were in the 5′ end, 3′ end, and the middle areas of the gene. One control vector contained the E. coli P-galactosidase gene in place of the modified odc gene was also made. This control vector contained no known sequences of significant homology with the rat genome. Rats were co-injected with fifty micrograms of the circular odc/P foot gene targeting vector and ten micrograms of plasmid DNA that encodes the P element transposase and circular negative control plasmids. After three weeks, the rats were killed and genomic DNA from the rats was prepared from a variety of tissues, some of which include, the liver and intestine. The DNA was subjected to PCR analyses, specifically primers that amplify the P foot and odc middle deficiency, and controls.

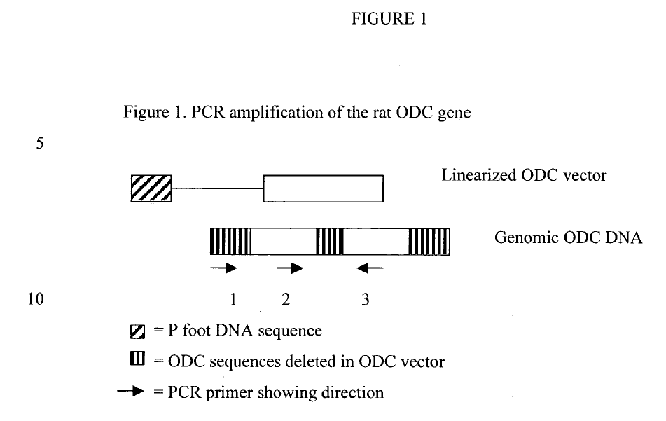

PCR of various samples of genomic DNA from rats administered controls using P foot or β-galactosidase-specific primers gave no products, therefore no detectable genomic integration of the control vectors had occurred. PCR of various samples of genomic DNA from rats administered the gene targeting vector with the modified odc gene revealed that the modified odc gene had integrated into the rat genome. Furthermore, no amplification could be detected using P foot specific primers in these samples, suggesting that the odc gene had integrated by a homologous recombination mechanism. Further proof was obtained for homologous recombination as illustrated in FIG. 1.

Shown in FIG. 1 is a schematic of the linearized ODC vector and the normal genomic ODC gene. The ODC gene was engineered to have three small deletions prior to its cloning into the ODC vector and these deletions are shown by vertical lines in the genomic ODC gene. Genomic DNA was isolated from tissues in rats that were injected with the above shown ODC vector. Using the PCR along with primers 2 and 3, the genomic DNA was tested for integration of the ODC vector in a variety of tissues. The PCR reaction detected a strong normal genomic ODC gene band of 500 bp in length. However, in many of the tissues only from injected animals (e.g., brain, testis, ovary, tail, liver, lung, heart, intestine, spleen to name a few), including some of their offspring, an additional band of 200 bp in length was detected (the size of the band for the ODC vector). The DNA samples that showed a band that indicated the presence of the ODC vector were then tested with PCR primers that specifically detect the P foot DNA. PCR analysis detected no presence of the P foot DNA. This finding suggests that the ODC vector was not randomly integrating into the rat genomic DNA, otherwise the PCR foot sequences would also be detectable. Finally, PCR using primers 1 and 3 performed on the samples showed that in the samples from the injected rats both the normal (genomic) and deleted (ODC vector) forms of the ODC gene were present. The only way to get the PCR amplification of the deleted form of the gene is if the ODC vector integrated into the ODC genomic gene in a homologous manner.

### EXAMPLE 2

### Gene Targeting of the Whey Associated Protein (WAP) Gene of Mice Using a Circular Gene Targeting Vector

To demonstrate this strategy is also successful in mice, a circular gene targeting vector is constructed. The circular vector contains a single Drosophila P element transposase cut site (P foot) and a fragment of altered mouse genomic DNA. The mouse Whey Associated Protein gene (WAP) is chosen because its entire sequence is known. In order to assay the integration of the vector, two small deletions are made in the WAP gene of the vector. The small deletions are at the 5′ end and the 3′ end of the gene. The E. coli β-galactosidase gene with a constitutively active mouse promoter is cloned into the middle of the modified WAP gene. Mice are co-injected with ten micrograms of the circular WAP gene targeting vector and two micrograms of plasmid DNA that encodes the P element transposase and circular negative control plasmids. After three months, genomic DNA is prepared from a variety of tissues from the injected animals as well as their offspring (e.g. blood, liver, lung, kidney). The DNA is subjected to PCR analyses, specifically primers that amplify the P foot, β-galactosidase gene, and both the WAP gene and β-galactosidase gene.

PCR of various samples of genomic DNA from mice administered controls using P foot or β-galactosidase-specific primers give no products, indicating that no detectable genomic integration of the control vectors has occurred. PCR of various samples of genomic DNA from mice administered the WAP gene targeting vector and controls show that the β-galactosidase gene is integrated into the genome and is located in the WAP genomic gene in the mouse genome. Finally, expression of the β-galactosidase gene is examined and expression is found in all tissues.

### EXAMPLE 3

The WAP vector is modified as follows: the β-galactosidase gene is removed, a small deletion in the middle of the WAP gene is made, and the P foot DNA sequences are deleted. This vector is linearized by subjecting the DNA to the restriction endonuclease, SspI. This linear DNA is injected into the mice and analyzed similarly as described in Example 2. The DNA integrates homologously into the WAP gene in all tested tissues in the injected animals as well as giving rise to progeny with a genetically altered WAP gene.

### EXAMPLE 4

Mice mutant for obesity were co-injected with 10 micrograms of an integration vector containing the normal form of the corresponding mutant gene and 1 microgram of the transposase DNA. A total of ten injections were carried out, one injection each week. Alternatively, a control vector containing no gene was also injected into the mutant mice. At the end of the five weeks the control mice showed an average increase in weight of 15% (approximately an 8 gram increase in weight/mouse). The mice injected with the integration vector that contained the normal obese gene sequences showed an average decrease in weight of 10% (approximately a 5 gram decrease in weight/mouse). As the integration vector carried the genomic DNA corresponding to exons 14-17 (and the introns as well for this region) of the obesity gene, only homologous integration would account for curing the obesity of the mouse. That is, the normal obese gene fragment could not rescue the phenotype if it were to randomly integrate since it contains only a small fraction of the gene and has no promoter and has no start site of translation, etc. The mutation in the obese mouse is known to occur between exons 15 and 16. This shows that not only is homologous integration occurring, but it is occurring enough to give a physiologic response. Furthermore, this particular obesity gene is needed to be produced in the brain, so this demonstrates that the technology is capable of passing through the blood:brain barrier.

### EXAMPLE 5

### Alternatives to a P Element Based Vector

A vector was constructed with the same deleted WAP gene as in Example 3 (see above). This time the vector backbone contained a single cut site for either a) Sleeping Beauty or b) other rare DNA cutting enzyme (e.g., PI-SCEI). Ten micrograms of these DNAs were injected along with the DNA encoding the cutting enzyme (one microgram of DNA for Sleeping Beauty enzyme and approximately 5 Units of PI-SCEI from New England Biolabs) into mice. After ten injections, spaced a week between each injection, no further procedures were done to the mice for one month. Then the mice were sacrificed and individual tissues/organs were collected and genomic DNA was prepared from each separate sample. Homologous integration was observed using these strategies, however it was at a frequency of integration far less than that observed for the P element based vector. This is probably due to the less efficient cutting by Sleeping Beauty. Also, for PI-SCEI the cutting could be occurring during the injection or the protein's ability to enter into cells is diminished or perhaps not enough enzyme was used.

### EXAMPLE 6

### Gene Therapy of Humans Using a Circular Gene Targeting Vector

To demonstrate this strategy is successful in humans, a circular gene targeting vector is constructed. The circular vector contains a single Drosophila P element transposase cut site (P foot) and a fragment of the normal human β-globin genomic gene. The human Beta Globin gene is chosen because its entire sequence is known and it is the cause of sickle cell anemia.

Patients with sickle cell anemia caused by a mutation in the chosen β-globin gene are administered 20 milligrams of the circular gene targeting vector with four milligrams of a P element transposase-expressing plasmid. After three weekly doses of the Beta Globin targeting construct, the blood morphology is examined to determine the presence of morphologically normal blood cells. Further, DNA analyses on genomic DNA isolated from periodic blood samples will determine that the Beta globin gene is being repaired by the gene targeting vector. Finally, the reduction of the symptoms of sickle cell anemia will be documented.

### EXAMPLE 7

### Homologous Integration Into Cells in Culture

Using a cell like human 293 kidney cells. Transfect the vector and linearizing enzyme DNAs into the cells via standard methods. Use one or multiple transfections to increase the efficiency as was done in animals (e.g., wait three to five days in between transfections for the residual DNA to clear through degradation or integrate into the genome). Cells can be assayed for homologous integration using standard techniques as described for whole animals. These include phenotype (cell morphology change, nutrient dependency or resistance, etc.), molecular (PCR, gene expression, etc), and other standard techniques (e.g., cell sorting, etc.). Cells can be manipulated externally from the animal just as they can be inside the animal. That is, cells in culture can be engineered to cause a deletion, DNA substitution, DNA insertion at a targeted locus. Concentrations of DNAs, transfection efficiencies, and number of repeat treatments will influence the efficiency of targeted integration just as observed when cells are treated in animals. These cells can then be used as assays for in vitro testing of gene function, protein production, drug testing, or similar other studies. Further, standard techniques can be used to turn these cells into whole animals, from standard embryonic stem cell growth by implanting this into an animal to grow into an embryo and develop into a living animal to donning techniques to turn single cells into animals.

It is evident from the above results and discussion that the subject invention provides efficient and highly effective reagents and methods to accomplish site specific integration of an exogenous nucleic acid into a target cell genome of a multicellular organism. Because the mechanism is homologous recombination, problems of random integration are avoided. Furthermore, the present invention provides for a significant increase in the efficiency of homologous recombination. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of homologously recombining an exogenous nucleic acid into a genome of a target cell, said method comprising:
introducing into said cell an integration vector comprising:
(a) a homologous recombination integrating element that comprises said exogenous nucleic acid flanked by sequences that provide for homologous recombination with said target genome; and
(b) a linearizing endonuclease recognition site that, in the presence of an endonuclease for the linearizing endonuclease site, is cleaved by said endonuclease to produce a linearized vector without significant cleavage of said target genome;
under conditions so that said integration vector is cleaved by said endonuclease to produce a linearized vector from which said integrating element homologously recombines into said target genome.

2. The method according to claim 1, wherein said integration vector is linearized by said endonuclease prior to introduction into said target cell.

3. The method according to claim 1, wherein said integration vector is linearized by said endonuclease after introduction into said target cell.

4. The method according to claim 1, wherein the endonuclease is a restriction enzyme.

5. The method according to claim 4, wherein the restriction enzyme is SspI.

6. The method according to claim 1, wherein said endonuclease is a recombinase.

7. The method according to claim 6, wherein said recombinase is a transposase.

8. The method according to claim 1, wherein said method further comprises introduction into said target cell said endonuclease or a nucleic acid comprising a coding sequence therefore.

9. The method according to claim 8, wherein said method further comprises introduction into said target cell a nucleic acid comprising a coding sequence for said endonuclease.

10. The method according to claim 9, wherein said coding sequence is not present on said integrating vector.

11. The method according to claim 9, wherein said coding sequence is present on said integrating vector.

12. The method according to claim 1, wherein integrating vector is introduced into said target cell in vitro.

13. The method according to claim 1, wherein said integrating vector is introduced into said target cell in vivo.

14. The method according to claim 1, wherein said integrating vector is introduced into said target cell ex vivo.

15. The method according to claim 1, wherein said target cell is a vertebrate cell.

16. The method according to claim 15, wherein said vertebrate cell is a mammalian cell.
